(19) United States
(12) Patent Application Publication
(10) Pub. No.: US 2008/0279932 A1
(43) Pub. Date: Nov. 13, 2008

(54) Compounds

(76) Inventors: Jean-Louis Reber, Kembs (FR); Edwin Bernard Villhauer, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/996,646

(22) PCT Filed: Aug. 2, 2006
(86) PCT No.: PCT/US06/30335

§ 371 Date: Jan. 24, 2008

Related U.S. Application Data
(60) Provisional application No. 60/705,592, filed on Aug. 4, 2005.

(51) Int. Cl. A61K 31/40; C07D 207/09; A61K 31/56; A61K 31/496; A61K 38/28; A61K 9/20; A61P 3/06; A61P 3/00; A61P 9/00; A61P 1/00; A61P 15/00; A61P 25/00; A61P 35/00; A61P 19/00

(52) U.S. Cl. 424/464; 548/528; 514/423; 514/171; 514/252.18; 514/4

(57) ABSTRACT

The present invention relates to novel salt forms of vildagliptin (LAF237), i.e. salt forms of(S)-1-[(3-hydroxy-1-adamantyl)amino]acetyl-2-cyano-pyrrolidine.

The present invention relates to novel salt forms of vildagliptin (LAF237), i.e. salt forms of (S)-1-[(3-hydroxy-1-adamantyl)amino]acetyl-2-cyano-pyrrolidine.

WO-A-00/34241 teaches that N-substituted-2-cyanopyrrolidines are inhibitors of DPP-IV, and are therefore useful in the treatment of non-insulin-dependent diabetes mellitus, arthritis, obesity, osteoporosis and further conditions of impaired glucose tolerance. The N-substituted-2-cyanopyrrolidines may exist in free base or acid addition salt form. A particular compound is (S)-1-[(3-hydroxy-1-adamantyl)amino]acetyl-2-cyano-pyrrolidine (“vildagliptin” or “LAF237”).

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention relates to novel salt forms of vildagliptin (LAF237), i.e. salt forms of (S)-1-[(3-hydroxy-1-adamantyl)amino]acetyl-2-cyano-pyrrolidine.

A first aspect of the invention is an acid addition salt of vildagliptin, or a salt mixture thereof. The acid may be any pharmaceutically acceptable acid, and examples of acid addition salts include 4-acetamidobenzoate, acetate, adipate, alginate, 4-aminosalicylate, ascorbate, aspartate, benzenesulfonate, benzoate, butyrate, camphorate, camphorsulfonate, carbonate, cinnamate, citrate, cyclamate, cyclopentanepropionate, decanoate, 2,2-dichloroacetate, digluconate, dodecylsulfate, ethane-1,2-disulfonate, ethanesulfonate, formate, fumarate, galactarate, gentisate, glucoheptanoate, gluconate, glucuronate, glutamate; glycerophosphate, glycolate, hemisulfate, heptanoate, hexanoate, hippurate, hydrochloride, hydrobromide, hydroiodide, 2-hydroxyethanesulfonate, isobutyrate, lactate, lactobionate, laurate, malate, maleate, malonate, mandelate, methanesulfonate, naphthalene-1,5-disulfonate, 2-naphthalenesulfonate, nicotinate, nitrate, octanoate, oleate, orotate, oxalate, 2-oxoglutarate, palmitate, pamoate, pectinate, persulfate, 3-phenylpropionate, phosphate, picrate, pidolate (L-pyroglutamate), pivalate, propionate, salicylate, sebacate, hydrogen sebacate, stearate, succinate, sulfate, tannate, tartrate, hydrogen tartrate, thiocyanate, tosylate, and undecanoate. In the case of polybasic acids, there are included acids in which all the acidic protons are removed as well as those in which one or, for example in the case of citrate, two protons are removed, as for example in the case of hydrogensulfate, hydrogenmalonate, hydrogenfumarate, hydrogenmalate, hydrogenmaleate, hydrogentartrate and hydrogengalactarate.

In certain embodiments of the disclosed salts, the salt is not one or more of a hydrochloride, methanesulfonate, sulfate, phosphate, citrate, lactate or acetate.

In one embodiment, there is provided an acid addition salt of vildagliptin in which the acid and the vildagliptin are substantially in 1:1 stoichiometry. The acid may be a monobasic or polybasic acid; exemplary polybasic acids are dibasic and tribasic.

The invention further provides salts of vildagliptin with polybasic acids in which the polybasic acid is substantially singly deprotonated.

Further included in the invention are the hydrochloride, sulfate or dicarboxylate (for example, a fumarate or malonate) salts of vildagliptin.

In another embodiment, there are provided carboxylic acid salts of vildagliptin. In one class of these salts, the acid is a polycarboxylic acid having two or more carboxylic acid groups. In a first sub-class, the polycarboxylic acids in these salts are substantially singly deprotonated, as for example in the case of a dicarboxylic acid salt having a 1:1 stoichiometry of vildagliptin and dicarboxylic acid. In a second sub-class, the polybasic carboxylic acid and the vildagliptin are in a substantially 1:1 stoichiometry, irrespective of the number of carboxylic acid groups in the acid.

Another aspect of the invention is a salt of the invention for therapeutic use.

Another aspect of the invention is a pharmaceutical formulation comprising a salt of the invention and, optionally, a pharmaceutically acceptable diluent or carrier.

A further aspect of the invention is a product i.e. combination product, comprising a salt of the invention and a therapeutic agent as a combined preparation for simultaneous, separate or sequential use in therapy.

Another aspect of the invention is the use of a salt of the invention, for the manufacture of a medicament for the treatment or prevention of a disease or condition selected from non-insulin-dependent diabetes mellitus, arthritis, obesity, allograft transplantation, calcitonin-osteoporosis, heart failure, impaired glucose metabolism or impaired glucose tolerance, neurodegenerative diseases, cardiovascular or renal diseases, and neurodegenerative or cognitive disorders, hyperglycemia, insulin resistance, lipid disorders, dyslipidemia, hyperlipidemia, hypertriglyceridemia, hypercholesterolemia, low HDL levels, high LDL levels, atherosclerosis, vascular restenosis, irritable bowel syndrome, inflammatory bowel disease, pancreatitis, retinopathy, nephropathy, neuropathy, syndrome X, ovarian hyperandrogenism (polycystic ovarian syndrome), type 2 diabetes, growth hormone deficiency, neutropenia, neuronal disorders, tumor metastasis, benign prostatic hypertrophy, gingivitis, hypertension and osteoporosis.

Another aspect of the invention is the use of a salt of the invention, for the manufacture of a medicament for producing a sedative or anxiolytic effect, attenuating post-surgical catabolic changes or hormonal responses to stress, reducing mortality and morbidity after myocardial infarction, modulating hyperlipidemia or associated conditions, or lowering VLDL, LDL or Lp(a) levels.

Another aspect of the invention is a method of treating or preventing a disease or condition in a patient, which comprises administering a therapeutically effective amount of a salt of the invention.

A further aspect of the invention is a process for preparing a salt of the invention in crystalline form, which comprises the steps of:

i) forming a solution comprising vildagliptin and a pharmaceutically acceptable acid,

ii) inducing crystallization of the salt, and

iii) recovering the crystalline vildagliptin salt.

In embodiments of the method, the vildagliptin and the acid are in 1:1 stoichiometry. Exemplary salts are as described above, for example the acid may be hydrochloric acid, sulfuric acid or a dicarboxylic acid. The dicarboxylic acid is preferably malonic acid or fumaric acid, i.e. the salt is preferably a malonate or fumarate respectively.

Compared with the free base, salts of the invention, or the amorphous forms, crystal forms, solvates, hydrates, and also the polymorphous forms thereof, advantageously have one or more improved properties.

The crystalline salts according to the invention may be more stable and of better quality than the free base, also during storage and distribution.

In addition, both the crystalline and the amorphous salts according to the invention may have a high degree of dissociation in water and thus substantially improved water solubility. These properties are of advantage, since on the one hand the dissolving process is quicker and on the other hand a smaller amount of water is required for such solutions. Salts of the invention may also lead to increased biological availability of the salts or salt hydrates in the case of solid dosage forms.

Improved physicochemical properties of certain salts or certain salt hydrates are of great importance both when produced as a pharmaceutically active substance and when producing, storing and applying galenic preparations. In this way, starting with improved constancy of the physical parameters, an even higher quality of the formulations can be guaranteed. High stability of a salts or salt hydrate also gives the possibility of attaining economic advantages by enabling simpler process steps to be carried out during working up. The high crystallinity of certain salt hydrates allows the use of a choice of analytical methods, especially the various X-ray methods, to permit a clear and simple analysis of their release to be made. This factor is also of great importance to the quality of the active substance and its galenic forms during production, storage and administration to the patients. In addition, complex provisions for stabilising the active ingredient in the galenic formulations can be avoided.

An essential feature for the quality of a pure active substance both for the physical-chemical procedures such as drying, sieving, grinding, and in the galenic processes which are carried out with pharmaceutical excipients, namely in mixing processes, in granulation, in spray-drying, in tabletting, is the water absorption or water loss of this active substance depending on temperature and the relative humidity of the environment in question. With certain formulations, free and bound water is without doubt introduced with excipients and/or water is added to the process mass for reasons associated with the respective formulation process. In this way, the pharmaceutical active substance is exposed to free water over rather long periods of time, depending on the temperature of the different activity (partial vapour pressure). The present salts may be advantageous in that they show no measurable water absorption or loss. This property is crucial in the final stages of chemical manufacture and also in practice in all galenic process stages of the different dosage forms. This exceptional stability similarly benefits the patients through the constant availability of the active ingredient.

Salts of the invention may also have an improved dissolving or compression hardness profile relative to the free base forms. Owing to their advantageous crystallinity, the salts may be suitable for pressing directly to form corresponding tablet formulations. An improved dissolving profile in tablet form may also be possible.

Salts of the invention may also have an improved pharmacokinetic profile, in particular they are particularly adapted to maintain a 24 hours inhibition of the dipeptidyl peptidase IV enzyme or at least 90% or 95% of inhibition of the dipeptidyl peptidase IV enzyme over 24 hours. Thus the Salts of the invention may be particularly adapted to develop pharmaceutical unit dosage form e.g. tablets, for a once a day administration to the patient. The AUC₀₋₂₄ (area under the plasma concentration-time curve from time zero to 24 hours [ng*hr/mL]) and/or the Cₘₐₓ (maximum plasma concentration) for vildagliptin can thus be improved and adapted for a once a day pharmaceutical unit dosage form.

Salts of the invention, may also have an improved stability when contained in a formulation comprising a further active ingredient. The salts may also have the advantage to avoid or reduce the degradation of the further active ingredient. Thus, the salts of the invention are particularly useful for combination therapy and to produce formulations comprising a further active ingredient e.g. a second antidiabetic agent such as metformin, pioglitazone, or rosiglitazone or an anti-hypertensive agent such as valsartan or in combination with a statin e.g. simvastatin or pravastatin.

The salts may also have the advantage that they are more efficacious, less toxic, longer acting, have a broader range of activity, more potent, produce fewer side effects, more easily absorbed than, or have other useful pharmacological properties over, compounds known in the prior art. Such advantages can also particularly occur during combination therapy with a further active ingredient e.g. a second antidiabetic agent such as metformin, pioglitazone, or rosiglitazone or an anti-hypertensive agent such as valsartan or in combination with a statin.

The extent of protection includes counterfeit or fraudulent products which contain or purport to contain a compound of the invention irrespective of whether they do in fact contain such a compound and irrespective of whether any such compound is contained in a therapeutically effective amount. Included in the scope of protection therefore are packages which include a description or instructions which indicate that the package contains a species or pharmaceutical formulation of the invention and a product which is or comprises, or purports to be or comprise, such a formulation or species.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

Further aspects and embodiments of the disclosure are set forth in the following description and claims.

### DESCRIPTION OF VARIOUS EMBODIMENTS

The following terms and abbreviations are used in this specification:

The term “Salts of the invention” as used herein includes amorphous forms, crystal forms, solvates, hydrates, and also the polymorphous forms of such a salt.

The term “crystalline form” as used herein includes reference to anhydrous crystalline forms, partially crystalline forms, mixture of crystalline forms, hydrate crystalline forms and solvate crystalline forms.

The term “hydrate” as used herein refers to a crystalline form containing one or more water molecules in a three-dimensional periodic arrangement.

The term “solvate” as used herein refers to a crystalline form containing one or more solvent molecules other than water in a three-dimensional periodic arrangement.

The term “a compound of the invention” refers to a salt of the invention.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

Methods for the synthesis of vildagliptin are described in WO-A-00/34241, the contents of which are incorporated herein by reference.

Any reference herein to the salts according to the invention is to be understood as referring also to the corresponding solvates, such as hydrates, and polymorphous modifications, and also amorphous forms, as appropriate and expedient. Salt mixtures are (i) single salt forms from different anions or (ii) mixtures of those single salt forms which exist, for example, in the form of conglomerates.

The salts of the invention preferably exist in isolated and essentially pure form, for example in a degree of purity of >95%, preferably >98%, more preferably >99%. The enantiomer purity of the salts according to the invention is preferably >98%, more preferably >99%.

The salts may be in crystalline, partially crystalline, amorphous or polymorphous form. The malonate and fumarate salt forms of vildagliptin are especially preferred. Typically, the stoichiometry of a salt of the invention is 1:1.

The salts may be dry. In embodiments, the salts are anhydrous.

The salts may be in solvate or hydrate form. Solvates and also hydrates of the salts according to the invention may be present, for example, as hemi-, mono-, di-, tri-, tetra-,penta-, hexa-solvates or hydrates, respectively. Solvents used for crystallisation, such a alcohols, especially methanol, ethanol, aldehydes, ketones, especially acetone, esters, e.g. ethyl acetate, may be embedded in the crystal grating. The extent to which a selected solvent or water leads to a solvate or hydrate in crystallisation and in the subsequent process steps or leads directly to the free base is generally unpredictable and depends on the combinations of process conditions and the various interactions between the free compound and the selected solvent, especially water. The respective stability of the resulting crystalline or amorphous solids in the form of salts, solvates and hydrates, as well as the corresponding salt solvates or salt hydrates, must be determined by experimentation. It is thus not possible to focus solely on the chemical composition and the stoichiometric ratio of the molecules in the resulting solid, since under these circumstances both differing crystalline solids and differing amorphous substances may be produced.

The description salt hydrates for corresponding hydrates may be preferred, as water molecules in the crystal structure are bound by strong intermolecular forces and thereby represent an essential element of structure formation of these crystals which, in part, are extraordinarily stable. However, water molecules may also exist in certain crystal lattices which are bound by rather weak intermolecular forces. Such molecules are more or less integrated in the crystal structure forming, but to a lower energetic effect. The water content in amorphous solids can, in general, be clearly determined, as in crystalline hydrates, but is heavily dependent on the drying and ambient conditions. In contrast, in the case of stable hydrates, there are clear stoichiometric ratios between the pharmaceutical active substance and the water. In many cases these ratios do not fulfil completely the stoichiometric value, normally it is approached by lower values compared to theory because of certain crystal defects. The ratio of organic molecules to water molecules for the weaker bound water may vary to a considerable extent, for example, extending over di-, tri- or tetra-hydrates. On the other hand, in amorphous solids, the molecular structure classification of water is not stoichiometric; the classification may however also be stoichiometric only by chance. In some cases, it is not possible to classify the exact stoichiometry of the water molecules, since layer structures form, so that the embedded water molecules cannot be determined in defined form.

Thus the invention also relates to the solid state physical properties of the compounds of the invention. These properties can be influenced by controlling the conditions under which a compound of the invention is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid or on the bioavailability of the drug. The rate of dissolution of an active ingredient in a patient's stomach fluid can have therapeutic consequences since it imposes an upper limit on the rate at which an orally-administered active ingredient can reach the patient's bloodstream.

For example, different crystal forms or amorphous form of the same drug may have substantial differences in such pharmaceutically important properties as dissolution rates and bioavailability. Likewise, different crystals or amorphous form may have different processing properties, such as hydroscopicity, flowability, and the like, which could affect their suitability as active pharmaceuticals for commercial production.

The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state 3C NMR spectrometry and infrared spectrometry. Method used to characterize the crystal form: IR, X-ray powder diffraction, melting point determination.

The crystalline forms of the invention may be identified and differentiated by X-ray diffraction and/or infrared spectroscopy, or any other method known in the art.

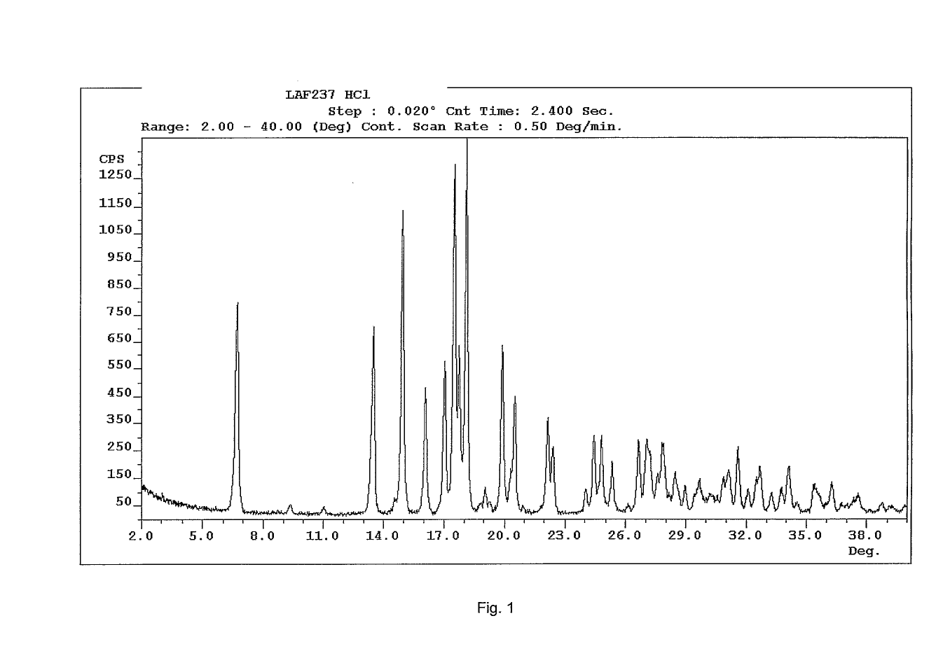

One embodiment of the present invention is a hydrochloride salt of vildagliptin in crystalline form, characterized by an X-ray diffraction pattern with peaks at about 15.0°, 17.6°, 18.2° and 19.9°+/−0.3° 2-theta, or with peaks at about 6.7°, 13.5°, 15.0°, 16.1°, 17.1°, 17.6°, 17.8°, 18.2°, 19.9°, 20.5°, 22.2° and 22.4°+/−0.3° 2-theta and preferably with peaks at about 6.7°, 13.5°, 15.0°, 16.1°, 17.1°, 17.6°, 17.8°, 18.2°, 19.9°, 20.5°, 22.2°, 22.4°, 24.5°, 24.8°, 25.4°, 26.7°, 27.1° and 27.9°+/−0.3° 2-theta. In a further embodiment of the present invention is a hydrochloride salt of vildagliptin in crystalline form, characterized by an X-ray diffraction pattern with peaks as essentially depicted on FIG. 1.

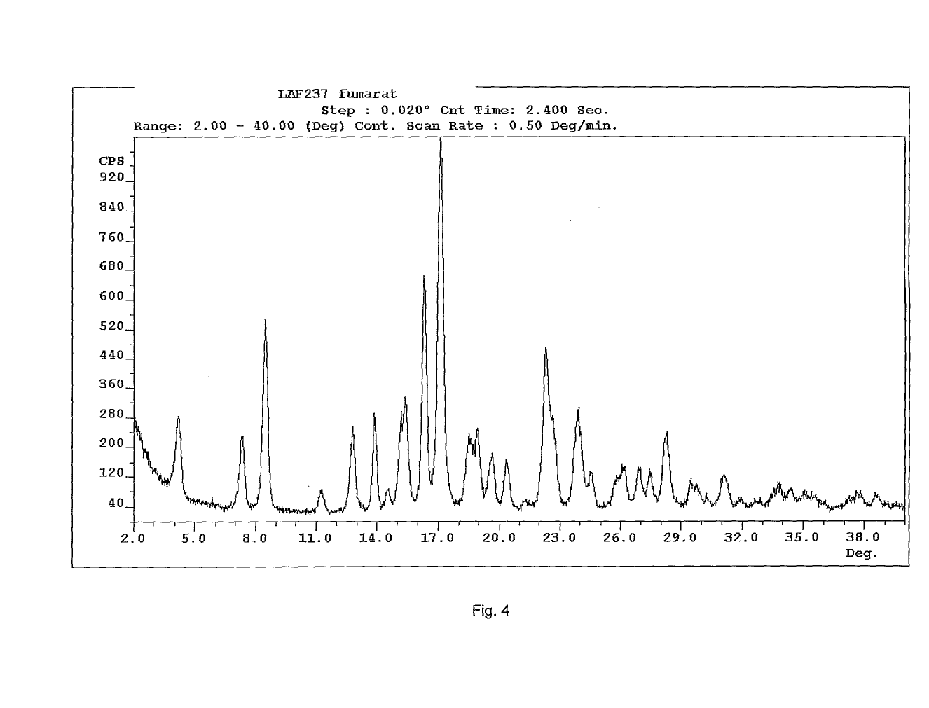

Another embodiment of the invention is a hydrogen fumarate salt of vildagliptin in crystalline form, characterized by an X-ray diffraction pattern with peaks at about 8.5°, 16.3°, 17.1° and 22.3°+/−0.3° 2-theta or with peaks at about 7.3°, 8.5°, 12.8°, 13.9°, 15.2°, 15.4°, 16.3°, 17.1°, 18.6°, 18.9°, 19.7°, 20.4°, 22.3° and 23.9°, 30 /−0.3° 2-theta preferably with peaks at about 4.2°, 7.3°, 8.5°, 11.25°, 12.8°, 13.9°, 15.2°, 15.4°, 16.3°, 17.1°, 18.6°, 18.9°, 19.7°, 20.4°, 22.3°, 23.9°, 24.6° and 25.8°+/−0.3° 2-theta. In a further embodiment of the present invention is a hydrogen fumarate salt of vildagliptin in crystalline form, characterized by an X-ray diffraction pattern with peaks as essentially depicted on FIG. 4.

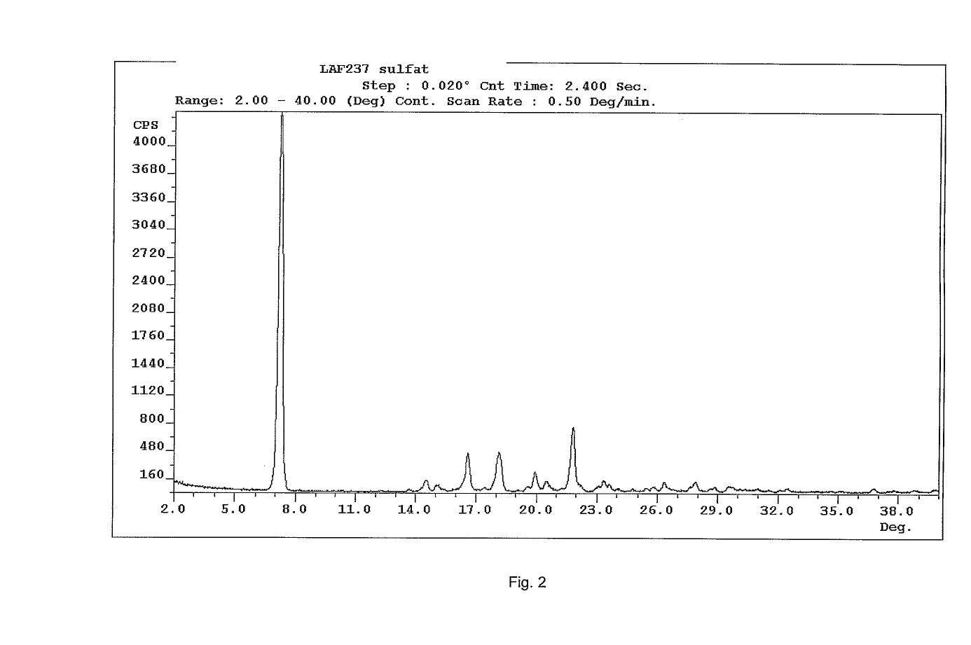

A further embodiment of the invention is a hydrogen sulfate salt of vildagliptin in crystalline form, characterized by an X-ray diffraction pattern with peaks at about 7.3°, 16.6°, 18.2°, and 21.8°+/−0.3° 2-theta or with peaks at about 7.3°, 14.5°, 15.2°, 16.6°, 18.2°, 20.0°, 20.5°, 21.8°, 23.1°, 23.4° and 23.6°+/−0.3° 2-theta preferably with peaks at about 7.3°, 14.5°, 15.2°, 16.6°, 18.2°, 19.6°, 20.0°, 20.5°, 21.8°, 23.1°, 23.4°, 23.6°, 26.3° and 27.9°+/−0.3° 2-theta. In a further embodiment of the present invention is a hydrogen sulfate salt of vildagliptin in crystalline form, characterized by an X-ray diffraction pattern with peaks as essentially depicted on FIG. 2.

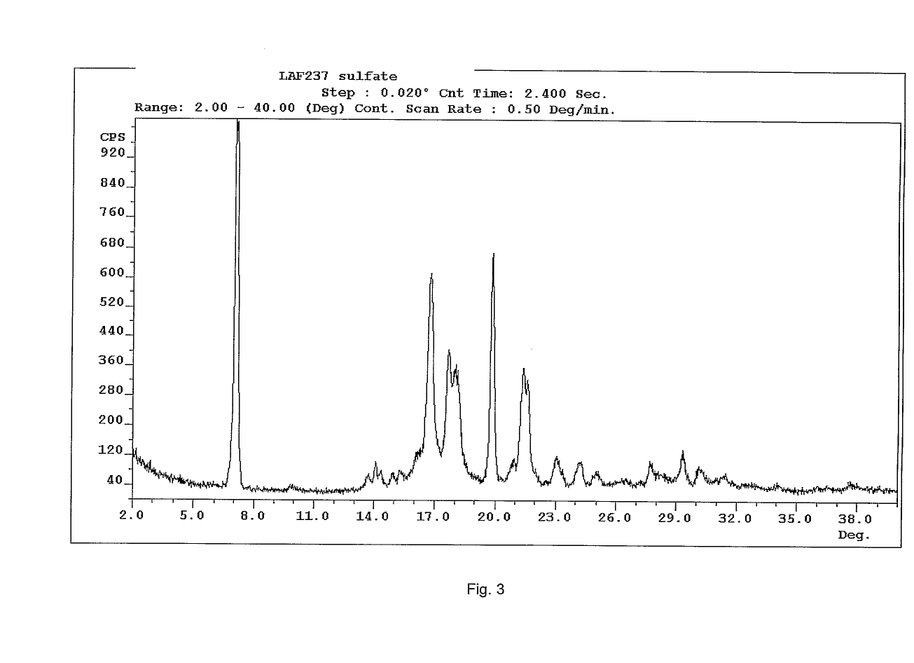

A further embodiment of the invention is a hydrogen sulfate salt of vildagliptin in crystalline form, characterized by an X-ray diffraction pattern with peaks at about 7.1°, 17.7°, 19.9°, and 21.6°+/−0.3° 2-theta or with peaks at about 7.1°, 14.1°, 16.8°, 17.7°, 18.0°, 19.9°, 21.6°, 23.1° and 24.3°+/−0.3° 2-theta preferably with peaks at about 7.1°, 14.1°, 16.3°, 16.8°, 17.7°, 18.0°, 19.9°, 20.1°, 21.4°, 21.6°, 23.1°, 24.3°, 27.8° and 29.4°+/−0.3° 2-theta. In a further embodiment of the present invention is a hydrogen sulfate salt of vildagliptin in crystalline form, characterized by an X-ray diffraction pattern with peaks as essentially depicted on FIG. 3.

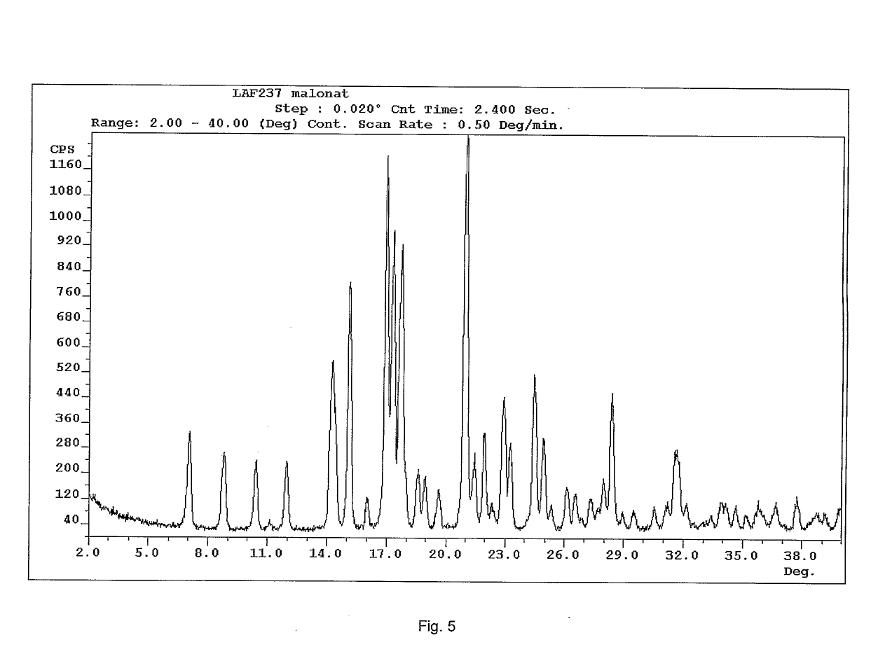

A further embodiment of the invention is a hydrogen malonate salt of vildagliptin in crystalline form, characterized by an X-ray diffraction pattern with peaks at about 15.1°, 17.0°, 17.3°, 17.8° and 21.0°+/−0.3° 2-theta or with peaks at about 7.1°, 8.8°, 10.4°, 12.0°, 14.3°, 15.1°, 17.0°, 17.3°, 17.8°, 18.6°, 19.0°, 21.0°, 22.0°, 22.9°, 23.3°, 24.5°, 25.0°, and 28.4°+/−0.3° 2-theta preferably with peaks at about 7.1°, 8.8°, 10.4°, 12.0°, 14.3°, 15.1°, 16.0°, 17.0°, 17.3°, 17.8°, 18.6°, 19.0°, 19.7°, 21.0°, 21.5°, 22.0°, 22.9°, 23.3°, 24.5°, 25.0°, 26.2°, 26.6°, 28.0°, 28.4° and 31.7°+/−0.3° 2-theta. In a further embodiment of the present invention is a hydrogen malonate salt of vildagliptin in crystalline form, characterized by an X-ray diffraction pattern with peaks as essentially depicted on FIG. 5.

In further embodiments, the present invention concerns crystalline forms of vildagliptin as characterized by the X-ray powder patterns provided (as substantially depicted) in FIGS. 1, 2, 3, 4 and 5, and Table 1.

As mentioned above, the crystalline forms of the invention may be characterized by X-ray diffraction. The X-ray diffraction patterns are unique for the particular crystalline forms. Each crystalline form exhibits a diffraction pattern with a unique set of diffraction peaks that can be expressed in 2 theta angles, d-spacing values and relative peak intensities. 2 Theta diffraction angles and corresponding d-spacing values account for positions of various peaks in the X-ray powder diffraction pattern. D-spacing values are calculated with observed 2 theta angles and copper K(al) wavelength using the Bragg equation, an equation well known to those of skill in the art.

A diffractometer measures the diffracted x-ray intensity (counts per second, cps) with respect to the angle of the X-ray source. Only crystalline samples diffract at well-defined angles, thus sharp peaks are observed depending on the nature of the crystal form. Each form will give a unique diffraction pattern. The intensity of the peaks depend on particle size and shape, thus it is a property of the batch not of the crystalline form. The diffraction peaks (pattern) defines the location of each atom within the molecule and defines the crystal symmetry and space group for the given crystal system.

It should be borne in mind that slight variations in observed 2 theta angles or d-spacing values are to be expected based on the specific diffractometer employed, the analyst, and the sample preparation technique. More variation is expected for the relative peak intensities.

Identification of the exact crystal form of a compound should be based primarily on observed 2 theta angles with no importance attributed to relative peak intensities. Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form.

Although 2 theta angles or d-spacing values are the primary methods of identifying the crystalline form, it may be desirable to also compare relative peak 5 intensities. As noted above, relative peak intensities may vary depending upon the specific diffractometer employed and the analyst's sample preparation technique. The peak intensities are reported as intensities relative to the peak intensity of the strongest peak. The peak intensities is usful for quality control but should not be used for crystal form identification.

X-ray diffraction provides a convenient and practical means for quantitative determination of the relative amounts of crystalline and/or amorphous forms in a solid mixture. X-ray diffraction is adaptable to quantitative applications because the intensities of the diffraction peaks of a given compound in a mixture are proportional to the fraction of the corresponding powder in the mixture. The percent composition of crystalline compound can be determined in an unknown composition.

Preferably, the measurements are made on the compound in solid powder form. The X-ray powder diffraction patterns of an unknown composition can be compared to known quantitative standards containing pure crystalline forms to identify the percent ratio of the crystalline form. This can be done by comparing the relative intensities of the peaks from the diffraction pattern of the unknown solid powder composition with a calibration curve derived from the X-ray diffraction patterns of pure known samples. The curve can be calibrated based on the X-ray powder diffraction pattern for the strongest peak from a pure crystalline sample.

In a further aspects, the present invention concerns a Malonate salt of vildagliptin in crystalline form, characterized by melting point of 170° C.+/−4° C. (obtained e.g. by Differential Scanning Calorimetry (DSC) method, 10 k/min).

In a further aspects, the present invention concerns a Sulfate I salt of vildagliptin in crystalline form, characterized by melting points of 130° C. and 196° C.+/−4° C. (obtained e.g. by Differential Scanning Calorimetry (DSC) method, 10 K/min).

In a further aspects, the present invention concerns a Fumarate salt of vildagliptin in crystalline form, characterized by melting point of 164° C.+/−4° C. (obtained e.g. by Differential Scanning Calorimetry (DSC) method, 10 K/min).

In a further aspects, the present invention concerns a Hydrochloride salt of vildagliptin in crystalline form, characterized by melting point of 234° C.+/−4° C. (obtained e.g. by Differential Scanning Calorimetry (DSC) method, 10 K/min).

In a further aspects, the present invention concerns a Sulfate II salt of vildagliptin in crystalline form, characterized by melting point of 191° C.+/−4° C. (obtained e.g. by Differential Scanning Calorimetry (DSC) method, 10 K/min).

In a further aspects, the present invention concerns a Bromide salt of vildagliptin in crystalline form, characterized by melting point of . . . ° C.+/−4° C. (obtained e.g. by Differential Scanning Calorimetry (DSC) method, 10 K/min).

Differential scanning calorimetry (DSC) curves are recorded using the Perkin Elmer or Mettler system. The powder shows a transition in the DSC thermogram at 147° C.+/−4° C. (method DSC, 2 C/min) corresponding to the melting of the substance.

In a further aspects, the present invention concerns the herein described salts of vildagliptin in crystalline form substantially characterized by the herein described X-ray diffraction pattern and DSC melting points.

### Synthesis

Salts of the present invention can be synthesized from the free base by conventional chemical methods. Generally, such salts can be prepared by reacting the free base form of the vildagliptin with the appropriate acid in water or in an organic solvent, or in a mixture of the two. The acid and the vildagliptin are combined in the desired stoichiometric ratio, for example 1:1; In many cases, nonaqueous media, for example ether, ethyl acetate, ethanol, isopropanol, or acetonitrile are used. As particular solvents may be mentioned organic solvents which are wholly or partly water miscible, for example an alkanol such as methanol, ethanol, propanol, isopropanol, butanol; acetone; methyl ethyl ketone; acetonitrile; DMF; DMSO In particular instances, the solvent comprises an alcohol, for example an alkanol, optionally in combination with water. Exemplary solvents are methanol, n-butanol, ethanol or isopropanol. The organic solvent, for example alcohol as described previously in this paragraph, is substantially dry in some embodiments.

Accordingly the salts or salt hydrates according to the invention can be obtained, for example, by neutralising the vildagliptin in free base form with an acid corresponding to the respective anion. The salt may be allowed or induced to crystallise. The salt may be allowed or induced to form an amorphous solid, optionally prior to crystallisation. The solid salt may be dried, e.g. by heating under reduced pressure.

Crystallisation may be effected in an organic solvent, particularly a water miscible organic solvent, water or an aqueous medium, which consists of water and at least one solvent that is miscible or partially miscible with water, i.e. not too non-polar, e.g. an alkanol such as methanol, ethanol, propanol, isopropanol, butanol, acetone, methyl ethyl ketone, acetonitrile, DMF, DMSO. The alkanol portion amounts for example to about 10 to 90, or 20 to 70, advantageously 30 to 50% by volume. For higher alkanols, the less polar solvent may also be present in lower concentrations. In a preferred variant, crystallisation may be optimised, e.g. accelerated, by adding at least one seed crystal.

Particularly exemplary solvents for crystallising the salts are n-butanol, ethanol and isopropanol.

By way of example, a method of preparing the salts, including amorphous or crystalline forms thereof, is as follows.

To form the salt, the process is carried out in a solvent system, in which the two reactants, namely the free base and the respective acid, are sufficiently soluble. It is expedient to use a solvent or solvent mixture in which the resulting salt is only slightly soluble or not soluble at all, in order to achieve crystallisation or precipitation. One variant for the salt according to the invention would be to use a solvent in which this salt is very soluble, and to subsequently add an anti-solvent, that is a solvent in which the resulting salt has only poor solubility, to the solution. A further variant for salt crystallisation consists in concentrating the salt solution, for example by heating, if necessary under reduced pressure, or by slowly evaporating the solvent, e.g. at room temperature, or by seeding with the addition of seeding crystals, or by setting up water activity required for hydrate formation. In yet another variant, an amorphous salt is obtained from the reaction solution, e.g. by removal of solvent, and the amorphous salt is redissolved in a crystallising solvent before crystallisation is induced, for example by allowing or causing cooling of a solution at elevated temperature, by concentrating the solution or by adding an anti-solvent.

The solvents that may be used are for example C₁-C₅ alkanols, preferably ethanol, isopropanol and n-butanol. Another alkanol to mention is methanol, although it has been found that salt crystallisation may not occur in methanol. As other solvents may be mentioned C₁-C₅ dialkylketones, preferably acetone. Any of the aforesaid solvents may be in admixture with water.

The antisolvents for salt crystallisation may be, for example, C₃-C₇ alkylnitriles, especially acetonitrile, esters, especially C₂-C₇ alkanecarboxylic acid C₁-C₅ alkylester, such as ethyl or isopropyl acetate, di-(C₁-C₅ alkyl)-ethers, such as tert-butylmethylether, furthermore tetrahydrofuran, and C₅-C₈ alkanes, especially pentane, hexane or heptane. Of these, tert-butylmethylether may particularly be mentioned.

The invention includes dry salts, for example prepared by drying the salt, suitably in crystalline form under reduced pressure and/or at elevated temperature (e.g. at 50-60° C. and optionally at ca. 15 mbar). The salt may be washed with an organic solvent, for example in the crystallising solvent (particularly in the case of crystals), prior to drying.

Particularly to be mentioned are methods for forming the salts of the invention by dissolving the vildagliptin and the acid in 1:1 stoichiometry in an alkanol, particularly methanol, n-butanol, ethanol or isopropanol. The solution may be at ambient temperature or elevated temperature (e.g. 40-75° C., more often 45-70° C.). If a crystallising solvent is chosen, the salt can be induced to form crystals in the solvent of the reaction mixture. As crystallising solvents may be mentioned:

| | |
|---|---|
| Vildagliptin hydrogen sulfate: | n-butanol |
| Vildagliptin hydrogen malonate: | ethanol |
| Vildagliptin hydrogen fumarate: | ethanol |
| Vildagliptin hydrochloride: | isopropanol. |

As methods of inducing crystallisation of the above salts from the corresponding solvents may be mentioned:

Vildagliptin hydrogen sulfate: seeding and cooling, e.g. to no more than 5° C., for example 0-3° C.,

Vildagliptin hydrogen malonate: seeding and then maintaining the mixture at e.g. no more than 25° C., optionally no more than 20° C., and suitably including cooling e.g. to no more than 5° C., for example 0-3° C.

Vildagliptin hydrogen fumarate: seeding and then maintaining the mixture at e.g. no more than 25° C., optionally no more than 20° C., and suitably including cooling e.g. to no more than 5° C., for example 0-3° C.

Vildagliptin hydrochloride: addition of anti-solvent (specifically tert-butyl-methyl ether), optionally combined with seeding and performed at a temperature of no more than 40° C., e.g. of 30° C., or below.

Vildagliptin bromide: seeding and then maintaining the mixture at e.g. no more than 25° C., optionally no more than 20° C., and suitably including cooling e.g. to no more than 5° C., for example 0-3° C.

If a non-crystallising solvent is used in the reaction (e.g. methanol, at least in the case of vildagliptin hydrogen sulfate), the amorphous salt may be redissolved in, and crystallised from, a crystallising solvent, e.g. the hydrogen sulfate may be crystallised from n-butanol.

Hydrates may be produced using a dissolving and crystallising process. The dissolving and crystallising process is characterised in that:
(i) the free base form and the appropriate acid are brought to a reaction in a preferably water-containing, organic solvent;
(ii) the solvent system is concentrated, for example by heating, if necessary under reduced pressure and by seeding with seeding crystals or by slowly evaporating, e.g. at room temperature, then crystallisation or precipitation is initiated; and
(iii) the salt obtained is isolated.

In the dissolving and crystallising process, the water-containing, organic solvent system is advantageously mixtures of alcohols, such as ethanol, and water; or alkylnitrile, especially acetonitrile, and water.

Alternatively, hydrates may be produced using a water-equilibrating crystallisation process. The equilibrating crystallisation process is characterised in that:
(i) the free base form and the appropriate acid are added to a water-containing organic solvent;
(ii) the solvent is concentrated, for example by heating, if necessary under reduced pressure or by slowly evaporating, e.g. at room temperature;
(iii) the residue of evaporation is equilibrated with the required amount of water by
   (a) suspending the residue of evaporation, which is advantageously still warm, and which still contains some water, in an appropriate solvent; or
   (b) by equilibrating the water excess in the solvent; wherein in a) and b), the existing or added water is present in a quantity in which the water dissolves in the organic solvent and does not form an additional phase; and
(iv) the salt obtained is isolated.

In the equilibration process, the water-containing organic solvent advantageously comprises mixtures of suitable alcohols, such as C₁-C₇ alkanols, especially ethanol, and water. An appropriate solvent for equilibration is, for example, an ester such as C₁-C₇ alkanecarboxylic acid-C₁-C₇ alkylester, especially ethyl acetate, or a ketone such as di-C₁-C₅-alkylketone, especially acetone. The equilibration process is notable for example for its high yields and outstanding reproducibility.

Other solvents suitable for use in the above procedures include esters, e.g. C₁-C₇ alkanecarboxylic acid-C₁-C₇ alkylesters, especially ethyl acetate, ketones, e.g. di-C₁-C₅-alkylketones, especially acetone, C₃-C₇ alkylnitriles, especially acetonitrile, or ethers, e.g. di-(C₁-C₅-alkyl)-ethers, such as tert.-butylmethylether, also tetrahydrofuran, or mixtures of solvents.

By using the dissolving and crystallising process, or the water-equilibrating crystallisation process, the defined hydrates, which are present in crystalline and in polymorphous forms, may be obtained reproducibly.

### Administration and Pharmaceutical Formulations

The compounds of the invention will normally be administered orally, intravenously, subcutaneously, buccally, rectally, dermally, nasally, tracheally, bronchially, by any other parenteral route, as an oral or nasal spray or via inhalation. The salts may be administered in a pharmaceutically acceptable dosage form. Depending upon the disorder and patient to be treated and the route of administration, the compositions may be administered at varying doses.

Typically, therefore, the pharmaceutical compounds of the invention may be administered orally or parenterally (“parenterally” as used herein, refers to modes of administration which include intravenous, intramuscular, intraperitoneal, intrasternal, subcutaneous and intraarticular injection and infusion) to a host to obtain an protease-inhibitory effect. In the case of larger animals, such as humans, the compounds may be administered alone or as compositions in combination with pharmaceutically acceptable diluents, excipients or carriers.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

In the treatment, prevention, control, amelioration, or reduction of risk of conditions which require inhibition of DPP-IV enzyme activity, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of the active ingredient for the symptomatic adjustment of the dosage to the patient to be treated. The compounds may be administered on a regimen of 1 to 4 times per day, preferably once or twice per day. The dosage regimen may be adjusted to provide the optimal therapeutic response.

According to a further aspect of the invention there is thus provided a pharmaceutical composition including a compound of the invention, in admixture with a pharmaceutically acceptable adjuvant, diluent or carrier.

Pharmaceutical compositions of this invention for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example. Prolonged absorption of the injectable pharmaceutical form may be brought about by the inclusion of agents (for example aluminum monostearate and gelatin) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are suitably made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, oral formulations contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g., sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g., chenodeoxycholic acid, cholic acid, deoxycholic acid, dehydrocholic acid and salts thereof, and glycine or taurine conjugate thereof); ionic surface active agents, such as sodium lauryisulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

The active compounds may also be in micro-encapsulated form, if appropriate, with one or more of the above-mentioned excipients. The active compounds may be in finely divided form, for example it may be micronised.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. The present compositions in liposome form can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Advantageously, the compounds of the invention may be orally active, have rapid onset of activity and low toxicity.

A compound of the invention is preferably in the form of a tablet, preferably one obtainable by direct compression.

One, two, three or more diluents can be selected. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g. may be present in an amount from about 15% to about 40% by weight of the composition. The preferred diluents include microcrystalline cellulose. Suitable microcrystalline cellulose will have an average particle size of from about 20 nm to about 200 nm. Microcrystalline cellulose is available from several suppliers. Suitable microcrystalline cellulose includes Avicel PH 101, Avicel PH 102, Avicel PH 103, Avicel PH 105 and Avicel PH 200, manufactured by FMC Corporation. Particularly preferred in the practice of this invention is Avicel PH 102. Preferably the microcrystalline cellulose is present in a tablet formulation in an amount of from about 25% to about 70% by weight.

Another diluent is lactose. Preferably, the lactose is ground to have an average particle size of between about 50 μm and about 500 μm prior to formulating. The lactose is present in the tablet formulation in an amount of from about 5% to about 40% by weight.

One, two, three or more disintegrants can be selected. Examples of pharmaceutically acceptable disintegrants include, but are not limited to, starches; clays; celluloses; alginates; gums; cross-linked polymers, e.g. cross-linked polyvinyl pyrrolidone, cross-linked calcium carboxymethylcellulose and cross-linked sodium carboxymethylcellulose; soy polysaccharides; and guar gum. The disintegrant, e.g. may be present in an amount from about 2% to about 20% by weight of the composition. Typical disintegrants include starch derivatives and salts of carboxymethylcellulose. Sodium starch glycolate is the preferred disintegrant for this formulation. Preferably the disintegrant is present in the tablet formulation in an amount of from about 0% to about 10% by weight, and can be from about 1% to about 4% by weight.

One, two, three or more lubricants can be selected. Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose.

The lubricant, e.g. may be present in an amount from about 0.1% to about 5% by weight of the composition; whereas, the glidant, e.g. may be present in an amount from about 0.1% to about 10% by weight. Such lubricants are commonly included in the final tablet mix in amounts usually less than 1% by weight. The lubricant component may be hydrophobic or hydrophilic. Examples of such lubricants include stearic acid, talc and magnesium stearate. Magnesium stearate reduces the friction between the die wall and tablet mix during the compression and ejection of the tablets. The preferred lubricant, magnesium stearate is also employed in the formulation. Preferably, the lubricant is present in the tablet formulation in an amount of from about 0.25% to about 6%. Other possible lubricants include talc, polyethylene glycol, silica and hardened vegetable oils. In an optional embodiment of the invention, the lubricant is not present in the formulation, but is sprayed onto the dies or the punches rather than being added directly to the formulation.

Other conventional solid fillers or carriers, such as, cornstarch, calcium phosphate, calcium sulfate, calcium stearate, magnesium stearate, stearic acid, glyceryl mono- and distearate, sorbitol, mannitol, gelatin, natural or synthetic gums, such as carboxymethyl cellulose, methyl cellulose, alginate, dextran, acacia gum, karaya gum, locust bean gum, tragacanth and the like, diluents, binders, lubricants, disintegrators, coloring and flavoring agents could optionally be employed.

Examples of pharmaceutically acceptable binders include, but are not limited to, starches; celluloses and derivatives thereof, e.g. microcrystalline cellulose, hydroxypropyl cellulose hydroxylethyl cellulose and hydroxylpropylmethyl cellulose; sucrose; dextrose; corn syrup; polysaccharides; and gelatin. The binder, e.g. may be present in an amount from about 10% to about 40% by weight of the composition.

Additional examples of useful excipients are described in the Handbook of pharmaceutical excipients, 3rd edition, Edited by A. H. Kibbe, Published by: American Pharmaceutical Association, Washington D.C., ISBN: 0-917330-96-X, or Handbook of Pharmaceutical Excipients (4^{th} edition), Edited by Raymond C Rowe—Publisher: Science and Practice which are incorporated herewith by reference.

Preferred formulations comprising the herein described salts and crystals are described in the patent application WO 2005/067976 and are incorporated herewith by reference.

The invention also provides compositions as described herein comprising between 20 and 200 mg, preferably between 20 and 160 mg, preferably between 25 and 150 mg, of a compound of the invention.

Preferred dosage for the free base form of vildagliptin is between 25 and 200 mg, most preferably between 50 and 150 mg or between 50 and 100 mg. Most preferably 50 mg or 100 mg or 150 mg. Thus, the preferred dosage form according to the present invention contains the corresponding mount of compound in the form of its salt i.e. same number of moles or mmoles (number of vildagliptin molecules). The final amount will depend on the weight of the corresponding salt.

The invention also provides compositions, pharmaceutical unit dosage forms, combinations, or uses, as described herein comprising between 20 and 200 mg, preferably between 20 and 160 mg, of a compound of the invention. Preferably between 20 and 200 mg of a compound of the invention is administered daily to the patient.

A formulation, combination, pharmaceutical unit dosage form, or indication as hereindescribed wherein the vildagliptin salt is selected from the group consisting of vildagliptin hydrogen malonate and vildagliptin hydrogen fumarate, or in any case a crystal form thereof.

The herein ratios have been obtained on a dry weight basis for the present compounds and diluents. The unit dosage form is any kind of pharmaceutical dosage form such as capsules, tablets, granules, chewable tablets, etc.

Preferably the present invention concerns a pharmaceutical composition comprising:
(a) 20-40% or 20-35% by weight on a dry weight basis of a compound of the invention;
(b) 40-95% preferably 62-78% by weight on a dry weight basis of a pharmaceutically acceptable diluent;
(c) 0-10% or 1-6% by weight on a dry weight basis of a pharmaceutically acceptable disintegrant; and optionally
(d) 0.1-10% or 0.25-6% by weight on a dry weight basis of a pharmaceutically acceptable lubricant.

Preferably the herein described compositions comprise;
i) one or two diluents selected from microcrystalline cellulose and lactose
ii) the two diluents microcrystalline cellulose and lactose,
iii) 25-70% preferably 35-55% by weight on a dry weight basis of a pharmaceutically acceptable microcrystalline cellulose, or
iv) 25-70% preferably 35-55% by weight on a dry weight basis of a pharmaceutically acceptable microcrystalline cellulose and 5-40% preferably 18-35% of lactose.

Most preferably the pharmaceutical composition comprises the pharmaceutically acceptable lubricant (d).

In the present application the reference to a pharmaceutically acceptable “disintegrant” or “diluent”, means at least one disintegrant or at least one diluent, a mixture of e.g. 2 or 3 disintegrants or 2 or 3 diluents is also covered.

Preferred diluents are microcrystalline cellulose or lactose or preferably a combination of microcrystalline cellulose and lactose, preferred disintegrant is sodium starch glycolate, and preferred lubricant is magnesium stearate.

The particular components in the preferred composition are the following:
(a) 20-35% by weight on a dry weight basis of a compound of the invention;
(b) 25-70% preferably 35-55% or 45-50% by weight on a dry weight basis of a pharmaceutically acceptable microcrystalline cellulose;
(c) 5-40% preferably 18-35% by weight on a dry weight basis of a pharmaceutically acceptable lactose;
(d) 0-10% preferably 1-4% by weight on a dry weight basis of a pharmaceutically acceptable sodium starch glycolate;
(e) 0.25-6% preferably 0.5-4 by weight on a dry weight basis of magnesium stearate.

Additional conventional excipients can optionally be added to the herein described formulations such as the conventional solid fillers or carriers described hereinabove.

The above described new compounds and compositions are particularly adapted for the production of pharmaceutical tablets e.g. compressed tablets or preferably direct compressed tablets, caplets or capsules and provides the necessary physical characteristics, dissolution and drug release profiles as required by one of ordinary necessary physical skill in the art. Therefore in an additional embodiment, the present invention concerns the use of any of the above-described compounds and formulations, for the manufacture of pharmaceutical tablets, caplets or capsules in particular for granulation, direct compression and dry granulation (slugging or roller compaction). In particular the tablets obtained with the above described compounds and formulations especially when processed in the form of tablets or direct compressed tablets, may have very low friability problems, low segregation of powders in the hopper during direct compression, good compressibility, cohesiveness and flowability of the powder blend, very good breaking strength, improved manufacturing robustness, optimal tablet thickness to tablet weight ratios, less water in the formulation especially directed compressed tablet, good Dispersion Disintegration time DT according to the British Pharmacopoeia 1988, good Dispersion Quality. The described advantages of the claimed compounds and compositions are also very useful for e.g. roller compaction or wet granulation or to fill capsules.

In the development of the herein described pharmaceutical compositions, the applicant has discovered that the compressed tablets especially direct compressed tablet is particularly advantageous if:
i) the particles comprising a compound of the invention have a particle size distribution of less than 250 μm, preferably between 10 to 250 μm, and/or
ii) the water content of the tablet at less than 10% after 1 week at 25° C. and 60% room humidity (RH), and/or
iii) tablet thickness to tablet weight ratio is of 0.002 to 0.06 mm/mg.

Thus in one embodiment (a), the present invention concerns pharmaceutical compositions/formulation, or compressed tablets preferably direct compressed pharmaceutical tablets, wherein the dispersion contains particles comprising a compound of the invention (salt or its crystal form) and wherein at least 40%, preferably 60%, most preferably 80% even more preferably 90% of the particle size distribution in the tablet is less than 250 μm or preferably between 10 to 250 μm. Preferably the particles contain one of the herein claimed salt crystal form.

The present invention concerns pharmaceutical compositions, or compressed tablets preferably direct compressed pharmaceutical tablets, wherein the dispersion contains particles comprising a compound of the invention, and wherein at least 40%, preferably 60%, most preferably 80% even more preferably 90% of the particle size distribution in the tablet is greater than 10 μm.

The term “wherein at least 40%, preferably 60%, most preferably 80% even more preferably 90%” means at least 40%, preferably at least 60%, most preferably at least 80%, even more preferably at least 90%. The term “wherein at least 25%, preferably 35% and most preferably 45%” means at least 25%, preferably at least 35% and most preferably at least 45%.

In particular the present invention concerns compressed tablets preferably direct compressed pharmaceutical tablets, wherein the dispersion contains particles comprising a compound of the invention, and wherein at least 25%, preferably 35% and most preferably 45% of the particle size distribution in the tablet is between 50 to 150 μm.

In another embodiment (b), this invention concerns a compressed tablet, preferably a direct compressed pharmaceutical tablet wherein the dispersion contains particles comprising a compound of the invention, and wherein tablet thickness to tablet weight ratio is of 0.002 to 0.06 mm/mg, preferably of 0.01 to 0.03 mm/mg.

The combination of the above embodiments (a) and (b), provide compressed tablets, preferably direct compressed tablets, with good compaction characteristics. Thus this invention concerns also a compressed tablet, preferably a direct compressed tablet wherein the dispersion contains particles comprising a compound of the invention, and wherein;
i) at least 40%, preferably 60%, most preferably 80% even more preferably 90% of the particle size distribution in the tablet is between 10 to 250 μm, and
ii) tablet thickness to tablet weight ratios is of 0.002 to 0.06 mm/mg or of 0.01 to 0.03 mm/mg, and optionally (preferably),
iii) the water content of the tablet is less than 10% after 1 week at 25° C. and 60% RH preferably wherein;
i) at least 25%, preferably 35% and most preferably 45% of the particle size distribution in the tablet is between 50 to 150 μm, and
ii) tablet thickness to tablet weight ratios is of 0.002 to 0.06 mm/mg or of 0.01 to 0.03 mm/mg, and optionally (preferably),
iii) the water content of the tablet is less than 10%, preferably 5%, after 1 week at 25° C. and 60% RH.

In a very preferred embodiment, the above described three embodiments, i.e. compressed tablets and direct compressed tablets contain the herein described pharmaceutical compositions

Preferably the particles comprise more than 60% of a compound of the invention, most preferably more than 90% or 95% and even more preferably more than 98% of the compound. Particles can alternatively be formed by microgranulation, a process well known in the art, and contain up to 40% of a pharmaceutically acceptable excipient.

It has been discovered that the selected particle size distribution of the active ingredient is particularly important to provide the best compaction of the tablets. Thus, in an additional preferred embodiment, the particle size distribution of the selected excipients (b), (c) and/or (d) is similar to the particle size distribution of the particles comprising the present compound. The term “similar” means that the particle size distribution of the excipient in the tablet is between 5 and 400 μm, or between 10 and 300 μm, preferably between 10 to 250 μm. The preferred excipients with an adapted particle size distribution can be chosen from e.g. Handbook of Pharmaceutical Excipients (4^{th} edition), Edited by Raymond C Rowe, Publisher: Science and Practice.

Particle size of drug is controlled by crystallisation, drying and/or milling/sieving. Particle size can also be comminuted using roller compaction and milling/sieving. Producing the right particle size is well known and described in the art such as in “Pharmaceutical dosage forms: volume 2, 2nd edition, Ed.: H. A. Lieberman, L. Lachman, J. B. Schwartz (Chapter 3: Siize Reduction)”.

Particle size distribution can be measured using Sieve analysis, Photon Correlation Spectroscopy or laser diffraction (international standart ISO 13320-1), or electronic sensing zone, light obstruction, sedimentation or microscopy which are procedures well known by the person skilled in the art. Sieving is one of the oldest methods of classifying powders by particle size distribution. Such methods are well known and described in the art such as in any analytical chemistry text book or by the United State Pharmacopeia's (USP) publication USP-NF (2004—Chapter 786—(The United States Pharmacopeial Convention, Inc., Rockville, Md.)) which describes the US Food and Drug Administration (FDA) enforceable standards. The used techniques are e.g. described in Pharmaceutical dosage forms: volume 2, 2nd edition, Ed.: H. A. Lieberman, L. Lachman, J. B. Schwartz is a good example. It also mentions (page 187) additional methods: Electronic sensing zone, light obstruction, air permeation, and sedimentation in gas or liquid.

In an air jet sieve measurement of particle size, air is drawn upwards, through a sieve, from a rotating slit so that material on the sieve is fluidised. At the same time a negative pressure is applied to the bottom of the sieve, which removes fine particles to a collecting device. Size analyses and determination of average particle size are performed by removal of particles from the fine end of the size distribution by using single sieves consecutively. See also “Particle Size Measurement”, 5th Ed. , p 178, vol. 1; T. Allen, Chapman & Hall, London, UK, 1997, for more details on this. For a person skilled in the art, the size measurement as such is thus of conventional character.

Water content of the tablet can be measured using Loss on drying method or Karl-Fischer method which are well known methods to the person skilled in the art (e.g. water content can be measured by loss on drying by thermogrametry). Such methods are well known and described in the art such as in any analytical chemistry text book (J. A. Dean, Analytical Chemistry Handbook, Section 19, McGraw-Hill, New York, 1995) or by the United State Pharmacopeia's (USP) publication USP-NF (2004) which describes the US Food and Drug Administration (FDA) enforceable standards ((2004—USP—Chapter 921).

Tablet thickness is measurable using a ruler, vernier caliper, a screw gauge or any electronic method to measure dimensions. We take the tablet thickness in mm and divide by tablet weight in mg to get the ratio. Such methods are well known and described in the art such as in any analytical chemistry textbook or by the United State Pharmacopeia's (USP) publication USP-NF (2004) which describes the US Food and Drug Administration (FDA) enforceable standards.

A further advantage of the formulations and tablets according to invention is that because the characteristics of the compounds of the invention, the resulting tablet will have a lower dissolution time and thus the drug may be absorbed into the blood stream much faster. Furthermore the fast dispersion times and relatively fine dispersions obtained with compounds of the invention are also advantageous for swallowable tablets. Thus formulations and tablets according to the invention can be presented both for dispersion in water and also for directly swallowing.

The Paddle method to measure the drug dissolution rate (% of release) is used with 1000 ml of 0.01N HCl. Such methods are well known and described in the art such as in any analytical chemistry text book or by the United State Pharmacopeia's (USP) publication USP-NF (2004—Chapter 711) which describes the US Food and Drug Administration (FDA) enforceable standards.

Processes for preparing the herein described tablets, or particles of the compounds of the invention are described in the patent application WO 2005/067976 which is incorporated herein by rference. The particles can be obtained by following the process of example 7 described in WO 2005/067976.

In another embodiment, the present invention covers capsule comprising the above described pharmaceutical compositions, and preferably wherein;
i) at least 60%, preferably 80% and most preferably 90% of the particles comprising a compound of the invention in the capsule have a particle size distribution between 10 to 500 μm,
ii) the water content of the tablet is less than 10% after 1 week at 25° C. and 60% RH.

More preferably capsule comprising the above described pharmaceutical compositions, and preferably wherein;
i) at least 40%, preferably 60%, most preferably 80% even more preferably 90% of the particles comprising a compound of the invention in the capsule have a particle size distribution of less than 250 μm preferably between 10 to 250 μm,
ii) the water content of the tablet is less than 5% after 1 week at 25° C. and 60% RH.

The final product is prepared in the form of tablets, capsules or the like by employing conventional tableting or similar machinery.

### Combination Therapies

The compounds of the invention may be administered in combination with one or more therapeutic agents. Accordingly, the invention provides a pharmaceutical composition comprising an additional agent. The invention also provides a product i.e. combination product, comprising a compound of the invention and an agent; as a combined preparation for simultaneous, separate or sequential use in therapy.

In particular, a composition or product of the invention may further comprise a therapeutic agent selected from anti-diabetic agents, hypolipidemic agents, anti-obesity or appetite-regulating agents, anti-hypertensive agents, HDL-increasing agents, cholesterol absorption modulators, Apo-A1 analogues and mimetics, thrombin inhibitors, aldosterone inhibitors, inhibitors of platelet aggregation, estrogen, testosterone, selective estrogen receptor modulators, selective androgen receptor modulators, chemotherapeutic agents, and 5-HT₃ or 5-HT₄ receptor modulators; or pharmaceutically acceptable salts or prodrugs thereof.

Examples of anti-diabetic agents include insulin, insulin derivatives and mimetics; insulin secretagogues, for example sulfonylureas (e.g. glipizide, glyburide or amaryl); insulinotropic sulfonylurea receptor ligands, for example meglitinides (e.g. nateglinide or repaglinide); insulin sensitisers, for example protein tyrosine phosphatase-1B (PTP-1B) inhibitors (e.g. PTP-112); GSK3 (glycogen synthase kinase-3) inhibitors, for example SB-517955, SB-4195052, SB-216763, NN-57-05441 or NN-57-05445; RXR ligands, for example GW-0791 or AGN-194204; sodium-dependent glucose cotransporter inhibitors, for example T-1095; glycogen phosphorylase A inhibitors, for example BAY R3401; biguanides, for example metformin; alpha-glucosidase inhibitors, for example acarbose; GLP-1 (glucagon like peptide-1), GLP-1 analogues and mimetics, for example exendin-4; DPPIV (dipeptidyl peptidase IV) inhibitors, for example DPP728, MK-0431, saxagliptin or GSK23A; AGE breakers; and thiazolidone derivatives, for example glitazone, pioglitazone, rosiglitazone or (R)-1-{4-[5-methyl-2-(4-trifluoromethyl-phenyl)-oxazol-4-ylmethoxy]-benzenesulfonyl}-2,3-dihydro-1H-indole-2-carboxylic acid (compound 4 of Example 19 of WO 03/043985) or a non-glitazone type PPAR-agonist (e.g. GI-262570); or pharmaceutically acceptable salts or prodrugs thereof.

Examples of hypolipidemic agents include 3-hydroxy-3-methyl-glutaryl coenzyme A (HMG-CoA) reductase inhibitors, for example lovastatin, pitavastatin, simvastatin, pravastatin, cerivastatin, mevastatin, velostatin, fluvastatin, dalvastatin, atorvastatin, rosuvastatin or rivastatin; squalene synthase inhibitors; FXR (farnesoid X receptor) ligands; LXR (liver X receptor) ligands; cholestyramine; fibrates; nicotinic acid; and aspirin; or pharmaceutically acceptable salts or prodrugs thereof.

Examples of anti-obesitylappetite-regulating agents include phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine and cannabinoid receptor antagonists e.g. rimonabant; or pharmaceutically acceptable salts or prodrugs thereof.

Examples of anti-hypertensive agents include loop diuretics, for example ethacrynic acid, furosemide or torsemide; diuretics, for example thiazide derivatives, chlorithiazide, hydrochlorothiazide or amiloride; angiotensin converting enzyme (ACE) inhibitors, for example benazepril, captopril, enalapril, fosinopril, lisinopril, moexipril, perinodopril, quinapril, ramipril or trandolapril; Na-K-ATPase membrane pump inhibitors, for example digoxin; neutralendopeptidase (NEP) inhibitors, for example thiorphan, terteo-thiorphan or SQ29072; ECE inhibitors, for example SLV306; dual ACE/NEP inhibitors, for example omapatrilat, sampatrilat or fasidotril; angiotensin II antagonists, for example candesartan, eprosartan, irbesartan, losartan, telmisartan or valsartan; renin inhibitors, for example aliskiren, terlakiren, ditekiren, RO-66-1132 or RO-66-1168; b-adrenergic receptor blockers, for example acebutolol, atenolol, betaxolol, bisoprolol, metoprolol, nadolol, propranolol, sotalol or timolol; inotropic agents, for example digoxin, dobutamine or milrinone; calcium channel blockers, for example amlodipine, bepridil, diltiazem, felodipine, nicardipine, nimodipine, nifedipine, nisoldipine or verapamil; aldosterone receptor antagonists; and aldosterone synthase inhibitors; or pharmaceutically acceptable salts or prodrugs thereof.

Examples of cholesterol absorption modulators include Zetia® and KT6-971, or pharmaceutically acceptable salts or prodrugs thereof.

Examples of aldosterone inhibitors include anastrazole, fadrazole and eplerenone, or pharmaceutically acceptable salts or prodrugs thereof.

Examples of inhibitors of platelet aggregation include aspirin or clopidogrel bisulfate, or pharmaceutically acceptable salts or prodrugs thereof.

Examples of chemotherapeutic agents include compounds decreasing the protein kinase activity, for example PBGF receptor tyrosine kinase inhibitors (e.g. imatinib or 4-methyl-N-[3-(4-methyl-imidazol-1-yl)-5-trifluoromethyl-phenyl]-3-(4-pyridin-3-yl-pyrimidin-2-ylamino)-benzamide), or pharmaceutically acceptable salts or prodrugs thereof.

Examples of 5-HT₃ or 5-HT₄ receptor modulators include tegaserod, tegaserod hydrogen maleate, cisapride or cilansetron, or pharmaceutically acceptable salts or prodrugs thereof.

The weight ratio of the compound of the present invention to the further active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200.

Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

A combination as described hereinabove, comprising:
i) a vildagliptin salt selected from the group consisting of vildagliptin hydrogen malonate and vildagliptin hydrogen fumarate, or in any case a crystal form thereof, and
ii) a HMG-CoA reductase inhibitor preferably selected from the group consisting of simvastatin, pravastatin, and fluvastatin.

A combination as described hereinabove, comprising:
i) a vildagliptin salt selected from the group consisting of vildagliptin hydrogen malonate and vildagliptin hydrogen fumarate, or in any case a crystal form thereof, and
ii) an antidiabetic compound preferably selected from the group consisting of metformin, sulfonylureas, thiazolidones, and insulin.

A combination as described hereinabove, comprising:
i) a vildagliptin salt selected from the group consisting of vildagliptin hydrogen malonate and vildagliptin hydrogen fumarate, or in any case a crystal form thereof, and
ii) an antiobesity agent preferably selected from cannabinoid receptor antagonists such as rimonabant.

A combination as described hereinabove, comprising:
i) a vildagliptin salt selected from the group consisting of vildagliptin hydrogen malonate and vildagliptin hydrogen fumarate, or in any case a crystal form thereof, and
ii) an anti-hypertensive agent preferably selected from the group consisting of benazepril, valsartan, aliskiren amlodipine and hydrochlorothiazide.

### Use

Compounds of the invention may be useful in the therapy of a variety of diseases and conditions.

In particular, the compounds of the invention may be useful in the treatment or prevention of a disease or condition selected from non-insulin-dependent diabetes mellitus, arthritis, obesity, allograft transplantation, osteoporosis, heart failure, impaired glucose metabolism or impaired glucose tolerance, neurodegenerative diseases (for example Alzheimer's disease or Parkinson disease), cardiovascular or renal diseases (for example diabetic cardiomyopathy, left or right ventricular hypertrophy, hypertrophic medial thickening in arteries and/or in large vessels, mesenteric vasculature hypertrophy or mesanglial hypertrophy), neurodegenerative or cognitive disorders, hyperglycemia, insulin resistance, lipid disorders, dyslipidemia, hyperlipidemia, hypertriglyceridemia, hypercholesterolemia, low HDL levels, high LDL levels, atherosclerosis, vascular restenosis, irritable bowel syndrome, inflammatory bowel disease (e.g. Crohn's disease or ulcerative colitis), pancreatitis, retinopathy, nephropathy, neuropathy, syndrome X, ovarian hyperandrogenism (polycystic ovarian syndrome), type 2 diabetes, growth hormone deficiency, neutropenia, neuronal disorders, tumor metastasis, benign prostatic hypertrophy, gingivitis, hypertension and osteoporosis.

The compounds may also be useful in producing a sedative or anxiolytic effect, attenuating post-surgical catabolic changes or hormonal responses to stress, reducing mortality and morbidity after myocardial infarction, modulating hyperlipidemia or associated conditions; and lowering VLDL, LDL or Lp(a) levels.

The compounds may also be particularly useful for the treatment or prevention of neurodegenerative or cognitive disorders, because of a better brain tissue distribution. Transporting vildagliptin across the blood-brain barrier via a compound of the invention (salt form of vildagliptin) is useful for achieving efficacious treatment or prevention of neurodegenerative or cognitive disorders.

Thus the invention also concerns;
the use of the compounds of the invention for improving the concentration of active ingredient (i.e. vildagliptin or its salts) in the brain tissues i.e. to improve the capability of crossing the blood-brain barrier,
the use of the compounds of the invention for transporting vildagliptin across the blood-brain barrier,
a method for transporting vildagliptin across the blood-brain barrier, wherein the patient is administered with a therapeutically effective amount of a compounds of the invention.

Use as hereinabove described wherein the vildagliptin salt is selected from the group consisting of vildagliptin hydrogen malonate and vildagliptin hydrogen fumarate, or in any case a crystal form thereof.

### EXAMPLES

The following Examples illustrate the invention.

The following salts were used in the selection program:

| | | | | Dissociation |
|---|---|---|---|---|
| | Acid Molecular | Formula | | Constant |
| Acid | Weight | Mass | Equivalents | (pKa's) |
| LAF237 | — | 303.4 | — | 7.86 |
| (free base) | | | | |
| hydrochloride | 36.45 | 339.9 | 1 | −6.1 |
| bromide | 80.92 | 384.3 | 1 | |
| benzoate | 122.12 | 425.5 | 1 | 4.2 |
| fumarate | 116.07 | 419.5 | 1 | 3.03/4.47 |
| malonate | 104.06 | 407.5 | 1 | 2.83/5.69 |
| maleate | 116.07 | 419.5 | 1 | 1.83/6.07 |
| tartarate | 150.09 | 453.5 | 1 | 2.98/4.34 |
| citrate | 192.12 | 495.5 | 1 | 3.06/4.74/6.40 |
| Oxalat | | | 1 | |
| Gentisate | | | 1 | |
| succinate | 118.09 | 421.5 | 1 | 4.16/5.16 |
| acetate | 60.05 | 363.5 | 1 | 4.76 |
| lactate | 90.08 | 393.5 | 1 | 3.08 |
| phosphate | 98.00 | 401.4 | 1 | 2.15/7.20/12.38 |

Preparation of LAF237 salts.

### Procedure:

1. 90.9 mg of drug substance is dissolved in 2 ml ethanol at 40° C.
2. Equimolar quantity of counter ion is dissolved in ethanol at 40° C.
3. The two solutions are mixed.

### Example 1

### Hydrochloride Salt of Vildagliptin

4.0 g LAF237 base (13.18 mmoles) was dissolved in 24 ml isopropanol at 70° C. Then 1.36 g hydrochloric acid (37% solution in water) (13.80 mmoles) were added dropwise over ca. 5 minutes. The solution was allowed to cool down. Seeding at 30° C. was followed by the addition of 5 ml tert-butyl-methyl ether at constant flow rate over ca. 10 minutes. The resulting thick suspension was stirred at room temperature for 3 hours and then filtered. The crystals were washed with 10 ml isopropanol and dried at 60° C./15 mbar for 20 hours.
Yield: 4.40 g white powder (93.8%)
Elementary analysis:
Calc.: 60.08% C, 7.71% H, 12.36% N, 9.42% 0, 10.43% Cl
Found: 60.03% C, 7.88% H, 12.33% N, 9.69% 0, 10.36% Cl

### Example 2

### Hydrogen Sulfate Salt (I) of Vildagliptin

0.614 g LAF237 base (2.023 mmoles) and 0.209 g sulfuric acid (assay 95%) (2.023 mmoles) were dissolved in 10 ml methanol at room temperature. The resulting solution was concentrated by 40° C. in vacuo. 0.50 g of the obtained amorphous residue was then dissolved in 5 ml n-butanol at 50° C. The solution was allowed to cool down under stirring. Crystallization slowly took place. The suspension was stirred for 19 h at room temperature and filtered. The crystals were washed with 2 ml n-butanol and dried at 50° C./ca. 15 mbar for 20 h.
Yield: 0.41 g of the title compound was obtained.
Elementary analysis:
Calc.: 50.86% C, 6.78% H, 10.47% N, 7.99% S, 23.91% O
Found: 50.64% C, 6.68% H, 10.44% N, 7.81% S, 23.97% O

### Example 3

### Hydrogen Sulfate Salt (II) of Vildagliptin

13.0 g LAF237 base (42.84 mmoles) was dissolved in 120 ml n-butanol at 60° C. 4.33 g sulfuric acid (assay 95%) (41.94 mmoles) was then dropwise added over five minutes: The resulting solution was allowed to slowly cool down. Crystallization took place after seeding at 32° C. The suspension was stirred 5 hours at room temperature and then cooled to 3° C. The mixture was further stirred at 0-3° C. for 17 hours. The suspension was filtered. The crystals were washed with 50 ml n-butanol of 0° C. and dried at 50° C./15 mbar for 20 h.
Yield: 13.70 g white powder (81.3%)
Elementary analysis:
Calc.: 50.86% C, 6.78% H, 10.47% N, 7.99% S, 23.91% O
Found: 50.89% C, 6.71% H, 10.43% N, 7.90% S, 24.02% O

### Example 4

### Hydrogen Fumarate Salt of Vildagliptin

13.0 g LAF237 base (42.84 mmoles) and 4.88 g fumaric acid acid (41.99 mmoles) were dissolved in 150 ml ethanol at 50° C. The solution was allowed to cool down. Crystallization took place after seeding at 42° C. The suspension was stirred for 4 hours at room temperature and then one additional hour at ca. 3° C. The resulting precipitate was filtered. The collected crystals were washed with 50 ml cold ethanol and dried at 50° C./15 mbar for 20 hours.
Yield: 17.10 g (97.1%)
Elementary analysis:
Calc.: 59.87% C, 6.92% H, 9.88% N, 23.32% O
Found: 59.71% C, 6.97% H, 10.03% N, 23.43% O

### Example 5

### Hydrogen Malonate Salt of Vildagliptin

13.0 g LAF237 base (42.84 mmoles) and 4.37 g malonic acid (41.99 mmoles) were dissolved in 150 ml ethanol at 45° C. The solution was allowed to cool down. Crystallization took place after seeding at 33° C. The suspension was stirred for 4 hours at room temperature and then for one additional hour at ca. 3° C. The resulting precipitate was filtered. The collected crystals were washed with 50 ml cold ethanol and dried at 50° C./15 mbar for 20 hours.
Yield: 15.05 g white crystals (88%)
Elementary analysis:
Calc.: 58.95% C, 7.17% H, 10.31% N, 23.56% O
Found: 58.96% C, 7.16% H, 10.46% N, 23.71% O

### Example 6

### X-ray Diffraction

The structure of each of the crystals of Examples 1 to 5 was determined by X-ray diffraction. The powder diffractometer used was the Type XDS 2000 or X1, Scintag, Santa Clara, USA. Procedure: The test substance was placed on the specimen holder. The X-ray diffraction pattern is recorded between 2° and 35° (2 theta) with Cu Ka radiation.

The measurements were performed at about 45 kV and 40 mA under the following conditions:
Scan rate: 0.5° (2 theta)/min
Chopper increment: 0.02°
Slits (from left to right): 2, 3, 0.3, 0.2 mm

The positions of all the lines in the X-ray diffraction pattern of the test substance with those in the X-ray diffraction pattern of the reference substance were compared. The X-ray diffraction pattern of the test substance correspond to the reference substance if the positions and relative intensities of the strong and medium strong bands are congruous, and no additional peaks or amorphous background appears in comparison to the reference substance.

X-ray powder diffractograms of the crystals of Examples 1 to 5 are shown in FIGS. 1 to 5 respectively. A list of the significant bands is provided in Table 1.

TABLE 1

| HCl | | Sulfate I | | Sulfate II | | Malonate | | Fumarate | |
|---|---|---|---|---|---|---|---|---|---|
| Peak | | Peak | | Peak | | Peak | | Peak | |
| pos. | Rel. Int. | pos. | Rel. Int. | pos. | Rel. Int. | pos. | Rel. Int. | pos. | Rel. Int. |
| (Deg.) | (%) | (Deg.) | (%) | (Deg.) | (%) | (Deg.) | (%) | (Deg.) | (%) |
| 6.73 | 26.29 | 7.27 | 100.00 | 7.11 | 100.00 | 7.09 | 25.38 | 4.20 | 27.15 |
| 13.50 | 47.00 | 14.54 | 3.43 | 14.09 | 9.30 | 8.83 | 20.32 | 7.33 | 22.27 |
| 14.96 | 78.76 | 15.16 | 2.11 | 16.30 | 13.52 | 10.43 | 17.94 | 8.50 | 50.69 |
| 16.10 | 33.56 | 16.61 | 10.21 | 16.79 | 57.90 | 11.99 | 18.14 | 11.25 | 8.17 |
| 17.06 | 39.07 | 18.18 | 10.38 | 17.70 | 38.61 | 14.27 | 42.58 | 12.81 | 23.43 |
| 17.55 | 88.70 | 19.62 | 1.76 | 18.03 | 33.10 | 15.13 | 62.94 | 13.88 | 27.07 |
| 17.78 | 43.89 | 19.97 | 5.32 | 19.86 | 61.58 | 16.04 | 9.66 | 15.24 | 25.90 |
| 18.15 | 100.00 | 20.54 | 3.07 | 20.95 | 10.12 | 16.98 | 90.75 | 15.40 | 32.12 |
| 19.93 | 43.08 | 21.83 | 16.85 | 21.40 | 32.95 | 17.33 | 75.06 | 16.33 | 64.20 |
| 20.54 | 31.42 | 23.10 | 1.91 | 21.61 | 29.66 | 17.75 | 71.50 | 17.13 | 100.00 |
| 22.17 | 24.56 | 23.37 | 3.27 | 23.05 | 10.65 | 18.61 | 15.99 | 18.56 | 21.87 |
| 22.42 | 18.33 | 23.64 | 2.26 | 24.26 | 9.65 | 18.96 | 14.98 | 18.94 | 24.23 |
| 24.46 | 20.74 | 26.34 | 2.88 | 27.73 | 9.57 | 19.66 | 11.48 | 19.66 | 17.02 |
| 24.84 | 20.17 | 27.90 | 2.95 | 29.35 | 12.10 | 21.00 | 100.00 | 20.38 | 15.70 |
| 25.37 | 13.77 | | | | | 21.46 | 19.11 | 22.33 | 45.81 |
| 26.67 | 19.30 | | | | | 21.96 | 26.06 | 23.91 | 29.16 |
| 27.09 | 20.35 | | | | | 22.94 | 33.39 | 24.56 | 12.86 |
| 27.86 | 19.66 | | | | | 23.28 | 22.18 | 25.80 | 11.21 |
| | | | | | | 24.49 | 39.10 | | |
| | | | | | | 24.98 | 23.99 | | |
| | | | | | | 26.16 | 12.24 | | |
| | | | | | | 26.58 | 10.80 | | |
| | | | | | | 28.00 | 13.89 | | |
| | | | | | | 28.40 | 33.69 | | |
| | | | | | | 31.70 | 21.16 | | |

### Example 7

### Stability of Bulk Material with Excipients for 2 week at 50 C and 50 C/75% r.h.

### SUMMARY

All three salt forms show little difference in assay values between the t₀ and 50° C. samples. All forms show some instability under humid conditions regardless of the excipient mixture. In the presence of mixture A, all of the salt forms behaved similarly with respect to % total impurities; however, in terms of % loss in assay value, the free base exhibited large losses that can not be reconciled with the level of impurities. The free base in mixture A was repeated twice and in both cases there were anomalous results with an apparent lack of mass balance. This may be indicative of an extraction problem of the free base with some component(s) in mixture A, or possibly undetected impurities. In addition, transesterification reaction of the hydroxyl group of LAF237 with cutina, a triglyceride (hydrogenated castor oil) may also explain the low assay value. The higher reactivity of the free base could be due to higher mutual solublility of these two phases compared to the salts. Additional studies are required to confirm this hypothesis.

Procedure for Dry Mixtures: 25 mg of free base drug substance was weighed into sample tube and approximately 2.5 grams of mixture A, or 25 mg of Lactose was added to the tube. Weight adjustments were made for each salt.

One sample preparation per condition and one injection per sample.

Controls: A control of the drug substance with the excipients will be prepared and analyzed as a time zero data point and to test the efficiency of extracting the drug substance from the excipients.

HPLC Assay Results (External Standard)
After 2 weeks at indicated temperature/humidity condition

| Temp/ | | | | |
|---|---|---|---|---|
| humidity | | Free Base | Cl⁻ Salt | Fumarate Salt |
| t₀ | Bulk | 100.0 | 100.7 | 104.0 |
| 50° C. | Bulk | 98.6 | 100.5 | 99.0 |
| 50/75% | Bulk | 98.7 | 99.6 | 98.9 |
| t₀ | 1% in mixture A | 98.1* | 97.3 | 96.1 |
| | Dry Granulated | | | |
| 50° C. | 1% in mixture A | 94.2* | 96.6 | 94.6 |
| | Dry Granulated | | | |
| 50/75% | 1% in mixture A | 83.8* | 95.5 | 96.0 |
| | Dry Granulated | | | |
| t₀ | 50% in Lactose Dry | 100.7 | 100.8 | 99.5 |
| | Granulated | | | |
| 50° C. | 50% in Lactose Dry | 98.9 | 99.7 | 98.9 |
| | Granulated | | | |
| 50/75% | 50% in Lactose Dry | 96.5 | 98.7 | 98.2 |
| | Granulated | | | |

*The original assay value for the 50° C. sample of Free Base in Mixture A was not consistent with the purity levels of the 50/75 and t0 samples, nor to comparable samples of the other salts, so the Free Base in Mixture A was repeated for all conditions and the data in the table is from the second analysis.

Mixture A: (oral) mannitol/Avicel PH 102/Cutina HR 57:38:5 (m/m/m)

The above described experimental a better stability of the claimed HCl salt or Fumarate salt or crystal forms thereof than the free base of vildagliptin. The malonate salt can also show improved stability.

Example 7 is a non limitative example showing the advantage of the developed and claimed new salts and crystal forms thereof.

### Example 8

### Forced Decomposition

Summary: All salt forms showed good bulk stability with no significant losses in assay after the three days at 80° C. LAF237 also exhibited good stability under acidic conditions at room temperature. The free base in water, however, proved to be very unstable with complete degradation of the drug after 3 days. The free base in water is basic and degradation proceeds as if in a basic solution. The same major impurity was observed in the peroxide sample with only 3% of the LAF237 remaining after three days.

Racemization Study: The drug in the solid state for both the free base and chloride showed no sign of racemization. In solution the chloride salt in water at both ambient and 80° C. also showed no sign of racemization. The free base in water chemically degraded and detection of racemization was not possible.

NOTE: Dissolving the free base drug substance in water is not recommended because the free base drug substance is basic and will increase the pH of unbuffered solutions. Therefore, to avoid degradation always dissolve the free base drug substance in a buffered solution in the acidic range. Thus, the claimed LAF237 salts are e.g. much more adapted for the production of tablets by wet granulation process.

Procedure: Weigh 25 mg drug substance to a test tube and add 5 ml of appropriate solution.

One sample preparation per condition and one injection per sample.

Dilute sample to 1 mg/mL with water for HPLC analysis.

All salt forms were tested in bulk and water.

| | HPLC Assay Results (External Standard) | | | | | |
|---|---|---|---|---|---|---|
| | Free Base | | HCl Salt | | Fumaric acid Salt | |
| Test | ambient | 80° C. | ambient | 80° C. | Ambient | 80° C. |
| Conditions | (3-day) | (3-day) | (3-day) | (3-day) | (3-day) | (3-day) |
| H₂O | 98.4 | 7.5 | 99.9 | 95.3 | 101.9 | 98.9 |
| Bulk | 99.9 | 100.3 | 98.8 | 98.8 | 102.2 | 98.8 |

The chloride and fumarate salts remain stable in water at 80° C. after 3 days. The malonate salt can also show a better stability than the free base.

NOTE: The HPLC method used for the forced decomposition had a mobile phase with a pH of 2.5 in which case the acid degradation product elutes after the amide degradation product. If the newer gradient method is selected then the order of elution of the acid degradation product and the amide degradation product are reversed, with the acid degradation product eluting first about 1.8 minutes and the amide degradation product eluting second about 2.5 minutes. Only the extended stability (3 month) samples were analyzed with the gradient HPLC method.

1. A salt of vildagliptin and a pharmaceutically acceptable acid in a 1:1 stoichiometry.

2. The salt of claim 1 which is a 4-acetamidobenzoate, acetate, adipate, alginate, 4-aminosalicylate, ascorbate, aspartate, benzenesulfonate, benzoate, butyrate, camphorate, camphorsulfonate, carbonate, cinnamate, citrate, cyclamate, cyclopentanepropionate, decanoate, 2,2-dichloroacetate, digluconate, dodecylsulfate, ethane-1,2-disulfonate, ethanesulfonate, formate, fumarate, galactarate, gentisate, glucoheptanoate, gluconate, glucuronate, glutamate, glycerophosphate, glycolate, hemisulfate, heptanoate, hexanoate, hippurate, hydrochloride, hydrobromide, hydroiodide, 2-hydroxyethanesulfonate, isobutyrate, lactate, lactobionate, laurate, malate, maleate, malonate, mandelate, methanesulfonate, naphthalene-1,5-disulfonate, 2-naphthalenesulfonate, nicotinate, nitrate, octanoate, oleate, orotate, oxalate, 2-oxoglutarate, palmitate, pamoate, pectinate, persulfate, 3-phenylpropionate, phosphate, picrate, pidolate (L-pyroglutamate), pivalate, propionate, salicylate, sebacate, hydrogen sebacate, stearate, succinate, sulfate, tannate, tartrate, hydrogen tartrate, thiocyanate, tosylate, or undecanoate salt.

3. The salt of claim 1 which is a hydrochloride, sulfate or dicarboxylate salt of vildagliptin.

4. The salt of vildagliptin which is a 4-acetamidobenzoate, acetate, adipate, alginate, 4-aminosalicylate, ascorbate, aspartate, benzenesulfonate, benzoate, butyrate, camphorate, camphorsulfonate, carbonate, cinnamate, citrate, cyclamate, cyclopentanepropionate, decanoate, 2,2-dichloroacetate, digluconate, dodecylsulfate, ethane-1,2-disulfonate, ethanesulfonate, formate, fumarate, galactarate, gentisate, glucoheptanoate, gluconate, glucuronate, glutamate, glycerophosphate, glycolate, hemisulfate, heptanoate, hexanoate, hippurate, hydrochloride, hydrobromide, hydroiodide, 2-hydroxyethanesulfonate, isobutyrate, lactate, lactobionate, laurate, malate, maleate, malonate, mandelate, methanesulfonate, naphthalene-1,5-disulfonate, 2-naphthalenesulfonate, nicotinate, nitrate, octanoate, oleate, orotate, oxalate, 2-oxoglutarate, palmitate, pamoate, pectinate, persulfate, 3-phenylpropionate, phosphate, picrate, pidolate (L-pyroglutamate), pivalate, propionate, salicylate, sebacate, hydrogen sebacate, stearate, succinate, sulfate, tannate, tartrate, hydrogen tartrate, thiocyanate, tosylate, or undecanoate.

5. A hydrochloride, sulfate or dicarboxylate salt of vildagliptin.

6. The salt according to claim 1, wherein the salt is a hydrochloride salt.

7. The salt according to claim 6, wherein the salt is in crystalline form and is characterized by an X-ray diffraction pattern with peaks
i) at about 15.0°, 17.6°, 18.2° and 19.9°+/−0.3° 2-theta, or
ii) at about 6.7°, 13.5°, 15.0°, 16.1°, 17.1°, 17.6°, 17.8°, 18.2°, 19.9°, 20.5°, 22.2° and 22.4°+/−0.3° 2-theta, or
iii) at about 6.7°, 13.5°, 15.0°, 16.1°, 17.1°, 17.6°, 17.8°, 18.2°, 19.9°, 20.5°, 22.2°, 22.4°, 24.5°, 24.8°, 25.4°, 26.7°, 27.1° and 27.9°+/−0.3° 2-theta, or
iv) as essentially depicted on FIG. 1.

8. The salt according to claim 1, wherein the salt is a hydrogensulfate salt.

9. The salt according to claim 8, wherein the salt is in crystalline form and is characterized by an X-ray diffraction pattern with peaks
i) at about 7.3°, 16.6°, 18.2°, and 21.8°+/−0.3° 2-theta, or
ii) at about 7.3°, 14.5°, 15.2°, 16.6°, 18.2°, 20.0°, 20.5°, 21.8°, 23.1°, 23.4° and 23.6°+/−0.3° 2-theta, or
iii) at about 7.3°, 14.5°, 15.2°, 16.6°, 18.2°, 19.6°, 20.0°, 20.5°, 21.8°, 23.1°, 23.4°, 23.6°, 26.3° and 27.9°+/−0.3° 2-theta, or
iv) as essentially depicted on FIG. 2.

10. The salt according to claim 8, wherein the salt is in crystalline form and is characterized by an X-ray diffraction pattern with peaks
i) at about 7.1°, 17.7°, 19.9°, and 21.6°+/−0.3° 2-theta, or
ii) at about 7.1°, 14.1°, 16.8°, 17.7°, 18.0°, 19.9°, 21.6°, 23.1° and 24.3°+/−0.3° 2-theta, or
ii) at about 7.1°, 14.1°, 16.3°, 16.8°, 17.7°, 18.0°, 19.9°, 20.1°, 21.4°, 21.6°, 23.1°, 24.3°, 27.8° and 29.4°+/−0.3° 2-theta, or
iv) as essentially depicted on FIG. 3.

11. The salt according to claim 1, wherein the salt is a hydrogenfumarate salt.

12. The salt according to claim 11, wherein the salt is in crystalline form and is characterized by an X-ray diffraction pattern with peaks
i) at about 8.5°, 16.3°, 17.1° and 22.3°+/−0.3° 2-theta, or
ii) at about 7.3°, 8.5°, 12.8°, 13.9°, 15.2°, 15.4°, 16.3°, 17.1°, 18.6°, 18.9°, 19.7°, 20.4°, 22.3° and 23.9°+/−0.3° 2-theta, or
iii) at about 4.2°, 7.3°, 8.5°, 11.25°, 12.8°, 13.9°, 15.2°, 15.4°, 16.3°, 17.1°, 18.6°, 18.9°, 19.7°, 20.4°, 22.3°, 23.9°, 24.6° and 25.8°+/−0.3° 2-theta, or
iv) as essentially depicted on FIG. 4.

13. The salt according to claim 1, wherein the salt is a hydrogenmalonate salt.

14. The salt according to claim 13, wherein the salt is in crystalline form and is characterized by an X-ray diffraction pattern with peaks
i) at about 15.1°, 17.0°, 17.3°, 17.8° and 21.0°+/−0.3° 2-theta, or
ii) at about 7.1°, 8.8°, 10.4°, 12.0°, 14.3°, 15.1°, 17.0°, 17.3°, 17.8°, 18.6°, 19.0°, 21.0°, 22.0°, 22.9°, 23.3°, 24.5°, 25.0°, and 28.4°+/−0.3° 2-theta, or
ii) at about 7.1°, 8.8°, 10.4°, 12.0°, 14.3°, 15.1°, 16.0°, 17.0°, 17.3°, 17.8°, 18.6°, 19.0°, 19.7°, 21.0°, 21.5°, 22.0°, 22.9°, 23.3°, 24.5°, 25.0°, 26.2°, 26.6°, 28.0°, 28.4° and 31.7°+/−0.3° 2-theta, or
iv) as essentially depicted on FIG. 2.

15. The salt according to claim 1, in crystalline, partially crystalline, amorphous or polymorphous form.

16. A hydrochloride salt of vildagliptin, in crystalline, partially crystalline, amorphous or polymorphous form.

17. The salt according to claim 1, in the form of a solvate.

18. The salt according to any of claims claim 1, in the form of a hydrate, for example a tetrahydrate or hexahydrate.

19. The salt of claim 1 which is dry.

20. The salt of claim 19 which is anhydrous.

21. A solution comprising a salt of claim 1.

22. The solution of claim 21 which is non-aqueous.

23. The solution of claim 22 wherein the solvent is an alkanol.

24. The solution of claim 21 which is aqueous.

25. The salt according to claim 1 for use in therapy.

26. A pharmaceutical formulation comprising a salt of claim 1.

27. The formulation according to claim 26, which further comprises a pharmaceutically acceptable excipient or carrier.

28. The formulation according to claim 26, which further comprises a therapeutic agent selected from anti-diabetic agents, hypolipidemic agents, anti-obesity or appetite-regulating agents, anti-hypertensive agents, HDL-increasing agents, cholesterol absorption modulators, Apo-A1 analogues and mimetics, thrombin inhibitors, aldosterone inhibitors, inhibitors of platelet aggregation, estrogen, testosterone, selective estrogen receptor modulators, selective androgen receptor modulators, chemotherapeutic agents, and 5-HT₃ or 5-HT₄ receptor modulators; or pharmaceutically acceptable salts or prodrugs thereof.

29. The formulation according to claim 28, wherein the agent is tegaserod, imatinib, metformin, a thiazolidone derivative, a sulfonylurea receptor ligand, aliskiren, valsartan, orlistat or a statin, or pharmaceutically acceptable salts or prodrugs.

30. The formulation according to claim 28, wherein the agent is selected from the group consisting of valsartan, simvastatin, pravastatin, fluvastatin, insulin, pioglitazone, rosiglitazone, and rimonabant.

31. The formulation according to claim 26, comprising between 20 and 200 mg of a salt of any of claims 1 to 20.

32. The formulation according to claim 26, wherein the vildagliptin salt is selected from the group consisting of the hydrogen malonate salt and the hydrogen fumarate salt, or in any case a crystal form thereof.

33. The formulation according to claim 26, wherein the dispersion contains particles comprising a salt of any of claims 1 to 20 and wherein at least 40%, or at least 60%, or at least 80%, or at least 90% of the particle size distribution in the tablet is less than 250 μm or preferably between 10 to 250 μm.

34. The formulation according to claim 33 which is a compressed tablet or a direct compressed pharmaceutical tablet.

35. A product comprising a salt of claim 1 and an therapeutic agent selected from anti-diabetic agents, hypolipidemic agents, anti-obesity or appetite-regulating agents, anti-hypertensive agents, HDL-increasing agents, cholesterol absorption modulators, Apo-A1 analogues and mimetics, thrombin inhibitors, aldosterone inhibitors, inhibitors of platelet aggregation, estrogen, testosterone, selective estrogen receptor modulators, selective androgen receptor modulators, chemotherapeutic agents, and 5-HT₃ or 5-HT₄ receptor modulators; or pharmaceutically acceptable salts or prodrugs thereof, as a combined preparation for simultaneous, separate or sequential use in therapy.

36. The product according to claim 35 comprising between 20 and 200 mg of a salt of any of claims 1 to 20.

37. The product according to claim 35 wherein the vildagliptin salt is selected from the group consisting of the hydrogen malonate salt and the hydrogen fumarate salt, or in any case a crystal form thereof.

38-41. (canceled)

42. A method for transporting vildagliptin across the blood-brain barrier, wherein the patient is administered with a therapeutically effective amount of a salt of claim 1.

43. A method of treating or preventing a disease or condition in a patient, which comprises administering to a patient a therapeutically effective amount of a salt of claim 1.

44. The method according to claim 43, wherein the disease or condition is selected from non-insulin-dependent diabetes mellitus, arthritis, obesity, allograft transplantation, calcitonin-osteoporosis, heart failure, impaired glucose metabolism or impaired glucose tolerance, neurodegenerative diseases, cardiovascular or renal diseases, and neurodegenerative or cognitive disorders, hyperglycemia, insulin resistance, lipid disorders, dyslipidemia, hyperlipidemia, hypertriglyceridemia hypercholesterolemia, low HDL levels, high LDL levels, atherosclerosis, vascular restenosis, irritable bowel syndrome, inflammatory bowel disease, pancreatitis, retinopathy, nephropathy, neuropathy, syndrome X, ovarian hyperandrogenism (polycystic ovarian syndrome), type 2 diabetes, growth hormone deficiency, neutropenia, neuronal disorders, tumor metastasis, benign prostatic hypertrophy, gingivitis, hypertension and osteoporosis.

45. (canceled)

46. A process for preparing a salt of claim 1 in crystalline form, which comprises the steps of:
i) forming a solution comprising vildagliptin and a pharmaceutically acceptable acid,
ii) inducing crystallization of the salt, and
iii) recovering the crystalline vildagliptin salt.

47. The process according to claim 46, wherein the solvent is methanol, n-butanol, ethanol or isopropanol.

48. The process according to claim 46, wherein crystallization is induced by adding an anti-solvent to the solution.

49. The process according to claim 46, wherein crystallization is induced by cooling, optionally combined with seeding

50. The process according to claim 46, wherein crystallization is induced by recovering amorphous salt from the reaction solution, redissolving the salt in a crystallising solvent, and inducing crystallisation in said solvent.

51. The process according to claim 46 wherein the recovered salt is dried.

52. The process according to claim 51 wherein the recovered salt is dried by heating under reduced pressure.

53. (canceled)
